(19) United States
(12) Patent Application Publication
(10) Pub. No.: US 2023/0050200 A1
(43) Pub. Date: Feb. 16, 2023

(54) SKINCARE AND FACIAL SCANNING SYSTEMS AND METHODS FOR PROVIDING SKINCARE DEVICE CONNECTIVITY AND SETTING CONFIGURATIONS

(71) Applicant: The Procter & Gamble Company, Cincinnati, OH (US)

(72) Inventors: Amruta JONNALAGADDA, Cincinnati, OH (US); Bud MIYAHARA, Fairfield, OH (US)

(21) Appl. No.: 17/885,967

(22) Filed: Aug. 11, 2022

Related U.S. Application Data
(60) Provisional application No. 63/231,736, filed on Aug. 11, 2021.

(51) Int. Cl. G16H 20/30; G06V 40/16

(52) U.S. Cl. CPC G16H 20/30; G06V 40/171

(57) ABSTRACT

Skincare and facial scanning systems and methods are disclosed herein for providing skincare device connectivity and setting configurations. A skincare device comprising a sensor is configure to scan, and to deposit a cosmetic ink composition onto, the skin of a user. A skincare application (app), communicatively coupled to the skincare device, generates a user-specific electronic analysis based on use of the skincare device on skin of a face portion of the user, wherein at least a portion of the user-specific electronic analysis is configured for display on a graphic user interface. In some aspects, the skincare device comprises an identification certificate configured to uniquely identify the skincare device to the skincare app upon a connection between the skincare device and the skincare app. The connection may be a persistent connection maintaining connectivity between the skincare device and the skincare app for a plurality of uses of the skincare device.

### FIELD

The present disclosure generally relates to skincare and facial scanning systems and methods, and more particularly to, skincare and facial scanning systems and methods for providing skincare device connectivity and setting configurations.

### BACKGROUND

Personal care devices typically assist users in a variety of healthcare areas. For example, one healthcare area is skincare, where electromechanical devices may be configured to assist a user improve the look and/or appearance of his or her skin. Typically such devices comprise electrical and mechanical components that interact with the user's skin.

Such conventional devices in the field of skincare, however, fail to capture and report feedback of the device itself, even more so fail to capture data or insights regarding the device's application in a user-specific manner, for example, as the device is operated or applied to the user's specific skin comprising a specific skin type. This can be problematic where a user lacks feedback as to the proper operation, use, status, or otherwise settings of a given skincare device and with respect to the user's specific skin and/or specific skin type.

For the foregoing reasons, there is a need for skincare and facial scanning systems and methods for providing skincare device connectivity and setting configurations, as further described herein.

### SUMMARY

Generally, as described herein, skincare and facial scanning systems and methods are disclosed for providing skincare device connectivity and setting configurations. As described herein, an example skincare device comprises the OPTE skincare device as developed or offered by PROCTER & GAMBLE CO. and/or its subsidiaries. Generally, the OPTE skincare device is configured to camouflage hyperpigmentation (i.e., “skin spots”) by scanning a user's skin, detecting skin spots, and then correcting the detected skin spots with a precision deposit of a serum (e.g., a cosmetic ink composition as described herein).

As described in the inventive disclosure herein, a skincare device, such as the OPTE skincare device, is configured to collect data regarding device usage, settings, performance, and device health. The skincare device may comprise a component as part of a skincare and facial scanning system, which can be a networked based system, for providing skincare device connectivity and setting configurations. When connected to the skincare and facial scanning system, for example, the skincare device may be enhanced from feedback of usage data of one or more individual users. More generally, the skincare and facial scanning systems and methods, as described herein, disclose a connected and networkable skincare device that allows for an always-on, fully connected, and/or persistent feedback loop that provides a user with user-specific skincare device and usage analysis. The feedback loop also allows for the skincare and facial scanning system to provide new or incremental updates to the skincare device by pushing new software (e.g., firmware) to introduce new features or fixes to the device, such as introducing new or different operations of the device, which may be based on user-specific needs or preferences for operation of the skincare device.

More specifically, as described herein, a skincare and facial scanning system is configured to provide skincare device connectivity and setting configurations. The skincare and facial scanning system may comprise a skincare device comprising a sensor configured to scan human skin and one or more nozzles configured to deposit a cosmetic ink composition onto human skin. The skincare and facial scanning system may further comprise a skincare application (app) comprising computing instructions and communicatively coupled to the skincare device. The computing instructions of the skincare app, when executed by one or more processors, may cause the one or more processors to generate a user-specific electronic analysis based on use of the skincare device on skin of a face portion of the user. At least a portion of the user-specific electronic analysis may be configured for display on a graphic user interface.

In addition, as described herein, a skincare and facial scanning method is described for providing skincare device connectivity and setting configurations. The skincare and facial scanning method may comprise scanning, by a skincare device comprising a sensor, skin of a user. The skincare and facial scanning method may further comprise depositing, by the skincare device, a cosmetic ink composition onto the skin of the user. The skincare and facial scanning method may further comprise generating, by a skincare application (app) communicatively coupled to the skincare device, a user-specific electronic analysis based on use of the skincare device on skin of a face portion of the user. At least a portion of the user-specific electronic analysis may be configured for display on a graphic user interface.

Still further, a tangible, non-transitory computer-readable medium storing instructions for providing skincare device connectivity and setting configurations is disclosed. The instructions, when executed by one or more processors, may cause the one or more processors to scan, by a skincare device comprising a sensor, skin of a user. The instructions, when executed by one or more processors, may further cause the one or more processors to deposit, by the skincare device, a cosmetic ink composition onto the skin of the user. The instructions, when executed by one or more processors, may further cause the one or more processors to generate a user-specific electronic analysis based on use of the skincare device on skin of a face portion of the user. At least a portion of the user-specific electronic analysis may be configured for display on a graphic user interface.

The present disclosure relates to improvements to other technologies or technical fields at least because the present disclosure describes or introduces improvements to computing devices in the skincare field, whereby a skincare device, comprising computing components, is configured to be improved by being connected as part of a skincare and facial scanning system for providing skincare device connectivity and setting configurations. For example, operation, performance, and/or features of the skincare device may be improved or updated by firmware updates based on detection and data acquisition of one or more users interacting with the skincare device (e.g., applying the skincare device to user skin). Such operation, performance, and/or features include updates to the sensor, light, scan range, nozzles (e.g., nozzle firing sequence, amount, or operation), settings of the skincare device, and/or other properties or parameters of the skincare device. The operation, performance, and/or features include updates may also include updates for the skincare device to use less memory or processing power to preserve data and/or power (e.g., battery) consumption of the skincare device. The new features and/or updates may be delivered over a computer network to a connected skincare device. In some aspects, the new features and/or updates may be provided when the skincare device is not detected in a usage state.

Still further, the present disclosure relates to improvement to other technologies or technical fields at least because the present disclosure describes or introduces improvements to computing devices in the field of security and/or image processing, where, at least in some aspects, images or scans of users are captured without depicting personal identifiable information (PII) of the individual. For example, a scan of a user's skin may not be stored and/or transmitted across a computer network, but where operation and/or settings data of the device as the device moves across the use's skin may be stored and/or transmitted stored. Such limited data capture and/or storage eliminates the need of transmission of images of individuals or portions of individuals across a computer network (where such images may be susceptible of interception by third parties). Such features provide a security improvement, i.e., where the non-transmission or non-storage of PII (e.g., user skin images and/or scans) provides an improvement over prior systems because images of a user's skin, especially ones that could be transmitted over a network (e.g., the Internet), are more secure without including or proving PII information of an individual. Accordingly, the systems and methods described herein can operate without the need for such essential information, which provides an improvement, e.g., a security improvement, over prior systems. In addition, the ability of the system to operate without storage or transmission of images, at least in some aspects, allows the underlying system to store and/or process little or no (or at least smaller data size) images, which results in a performance increase to the underlying system as a whole because the limited or smaller data size images require less storage memory and/or processing resources to store, process, and/or otherwise manipulate the underlying skincare device and skincare and facial scanning system as a whole.

In addition, the present disclosure includes applying the certain of the aspects disclosed herein with, or by use of, a particular machine, e.g., a skincare device comprising a sensor configured to scan human skin and one or more nozzles configured to deposit a cosmetic ink composition onto human skin.

In addition, the present disclosure includes specific features other than what is well-understood, routine, conventional activity in the field, and that add unconventional steps that confine the claim to a particular useful application, e.g., skincare and facial scanning systems and methods for providing skincare device connectivity and setting configurations as described herein.

Advantages will become more apparent to those of ordinary skill in the art from the following description of the preferred aspects which have been shown and described by way of illustration. As will be realized, the present aspects may be capable of other and different aspects, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts a particular aspect of the disclosed system and methods, and that each of the Figures is intended to accord with a possible aspect thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present aspects are not limited to the precise arrangements and instrumentalities shown, wherein:

FIG. 1 is an exploded view of a skincare care device for use in depositing a cosmetic ink composition, in accordance with various aspects disclosed herein.

FIG. 2 is an exploded view of a cartridge containing the cosmetic ink composition as described for FIG. 1, and in accordance with various aspects disclosed herein.

FIG. 3 illustrates an example skincare and facial scanning system configured to provide skincare device connectivity and setting configurations, in accordance with various aspects disclosed herein.

FIG. 4 illustrates an example skincare and facial scanning method for providing skincare device connectivity and setting configurations, in accordance with various aspects disclosed herein.

FIG. 5A illustrates an example graphic user interface depicting a user-specific electronic analysis based on use of the skincare device as describe for FIG. 1, in accordance with various aspects disclosed herein.

FIG. 5B illustrates a further example graphic user interface depicting a user-specific electronic analysis based on use of the skincare device as describe for FIG. 1, in accordance with various aspects disclosed herein.

FIG. 5C illustrates an additional example graphic user interface depicting a user-specific electronic analysis based on use of the skincare device as describe for FIG. 1, in accordance with various aspects disclosed herein.

FIG. 6A illustrates an example graphic user interface depicting a recommendation for a product, in accordance with various aspects disclosed herein.

FIG. 6B illustrates an example graphic user interface depicting usage data defining use of the skincare device as captured over time, in accordance with various aspects disclosed herein.

FIG. 6C illustrates an example graphic user interface depicting a usage or device settings screen for editing usage or device settings data of the skincare device, in accordance with various aspects disclosed herein.

FIG. 7 illustrates an example graphic user interface as rendered on a display screen of a user computing device in accordance with various aspects disclosed herein.

FIG. 8 illustrates a further example graphic user interface as rendered on a display screen of a user computing device in accordance with various aspects disclosed herein.

FIG. 9 illustrates a further example graphic user interface as rendered on a display screen of a user computing device in accordance with various aspects disclosed herein.

FIG. 10 illustrates a further example graphic user interface as rendered on a display screen of a user computing device in accordance with various aspects disclosed herein.

The Figures depict preferred aspects for purposes of illustration only. Alternative aspects of the systems and methods illustrated herein may be employed without departing from the principles of the invention described herein.

### DETAILED DESCRIPTION OF THE INVENTION

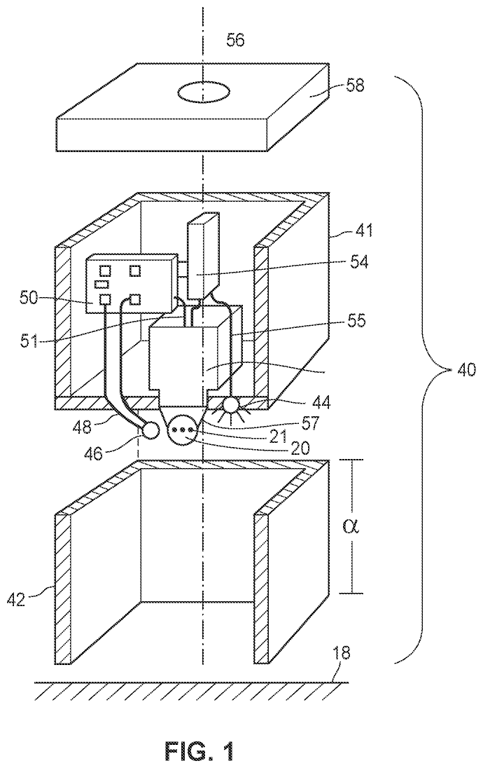

FIG. 1 is an exploded view of a skincare device 40 for use in depositing a cosmetic ink composition (e.g., a serum), in accordance with various aspects disclosed herein. Generally, as described herein, the cosmetic ink composition that can exhibit long-term particle suspension stability and a skincare device 40 for depositing the cosmetic ink composition. In one aspect, “stable white ink” can refer to an ink substantially free from particle settling, where no agitation or mixing is required to use the ink for its application.

In one aspect, the cosmetic ink composition described herein can be jetted onto any surface, preferably a keratinous surface including human skin and/or hair.

The cosmetic ink composition can comprise a unique combination of a rheology modifier and a (meth)acrylic acid homopolymer or a salt thereof that can suspend a particulate material having particles large enough to be visually perceptible, yet can still be j enable. Careful balancing of the agglomeration of the particles and viscosity of the cosmetic ink composition can be used to inhibit particle settling, providing particle suspension stability beyond which has been previously reported without hindering high frequency printing capabilities via thermal and/or piezo inkjet printheads. One advantage to this is that little to no shaking, and/or agitation of the cosmetic ink composition by the consumer or automated mechanical processes before and/or during printing is needed to re-disperse the particles. This can make the cosmetic ink composition more user-friendly as it does not require the consumer to perform an additional step to re-disperse the particles and/or can eliminate the need for agitation or rotation systems, including automated systems, within the printing device, cartridge, printer servicing station, and/or docking station.

Without being limited by theory, it is believed that the cosmetic ink composition can be stabilized using a (meth)-acrylic acid homopolymer or a salt thereof to minimize and/or prevent particle agglomeration and a rheology modifier to introduce a secondary structure and build viscosity to suspend the particles in a weak colloidal gel. The colloidal gel can be strong enough to hold the particles in suspension when not printing (thus inhibiting particle settling), but weak enough to break up during printing. An exemplary cosmetic ink composition is described in U.S. patent Ser. No. 16/262,967, which is incorporated by reference in its entirety herein.

With reference to FIG. 1, the cosmetic ink composition described herein can be applied to the skin using skincare device 40, which can be a hand-held personal care device or the like. An example skincare device comprises the OPTE skincare device as developed or offered by PROCTER & GAMBLE CO. and/or its subsidiaries. In addition, an exemplary skincare device or otherwise personal care device is described in U.S. Pat. No. 9,522,101, which is incorporated by reference in its entirety herein.

Skincare device 40 can analyze the skin of a user, identify a skin imperfection(s), and can deposit the cosmetic ink composition onto the identified skin imperfection(s) in order to hide and/or camouflage the skin imperfection(s).

In one aspect, the skincare device 40 can comprise a sensor configured to scan, capture, and/or otherwise take at least one image of skin and a processor configured to calculate the average background lightness value of the image on a grey scale (lightness value on a grey scale is herein referred to as “L value”). Further, from the same image, a local L value can be calculated for individual pixels or a group of pixels. The processor can then compare the local L value to the background L value to identify skin imperfections. When a skin imperfection is identified, the processor can activate one or more nozzles to fire and dispense, or otherwise deposit, the cosmetic ink composition onto the skin imperfection.

A skin imperfection is an area of skin where the absolute value of the difference between a local L value and the background L, this difference being defined as the measured delta L (“ΔL_{M}”), is greater than a predetermined set delta L (“ΔL_{S}”). The background L can be preset or calculated anywhere within the image. The image can be taken where the nozzles will fire the cosmetic ink composition. The background L can be the arithmetic average, median, or mean of a plurality of local L_{S}, which means the calculation can include all of the local L_{S} in the image, or a subset thereof.

With reference to FIG. 1 skincare device 40 comprises a physical spacer 42 positioned directly above skin surface 18. Physical spacer 42 has a set, predetermined height a such that when it contacts skin surface 18, the mechanical and electrical elements are all at a known distance from skin surface 18. In one aspect, the height a is from about 1 mm to about 20 mm, alternatively from about 3 mm to about 15 mm, alternatively from about 4 mm to about 10 mm.

The mechanical and electrical elements associated with skincare device 40 can include, but are not be limited to, light 44, sensor 46 (e.g., an image sensor), nozzle array 20 which is embedded on cartridge, and/or die 57 which is attached to cartridge 52. Cartridge die 57 can be made of silicon, glass, machinable glass ceramic, sapphire, alumina, printed wiring board substrates (for example, Liquid Crystal Polymer, polyimide etc.) within which nozzle array 20 can be formed. Nozzle array 20 can be in a linear configuration, multiple rows, off-set, sine wave, curved, circular, saw tooth arrangements, and combinations thereof. All of these elements can be enclosed within optional apparatus housing 41.

Light 44 can illuminate the area of skin surface 18 within physical spacer 42 such that sensor 46 has relatively constant illumination. Background lighting can affect sensor 46 as portions of physical spacer 42 lift off skin surface 18 and allow background light in and the illumination from light 44 to escape. Small deviations in illumination can be corrected for provided light 44 provides a relatively constant background illumination. In one aspect, physical spacer 42 can be opaque. Light 44 can be a LED, incandescent light, neon bulb based, or any other commercially available source of illumination. Light 44 can have constant illumination or adjustable illumination. For example, an adjustable light source might be useful if the background illumination is excessively bright or dark.

Sensor 46 can be any component that is capable of obtaining a visual property of an area of skin surface. Non-limiting examples of sensors can include optical sensors, image sensors, image capture devices, spectrophotometers, photonic measuring devices for wavelengths within the visible spectrum as well as those wavelengths above and below the visible spectrum which could measure sub-surface features, and combinations thereof. The image capture device can be any of a variety of commercially available devices such as a simple camera or a digital complementary metal-oxide semiconductor (CMOS) camera chip. In one aspect, the image capture device can be a camera and the images can be taken or converted to a standard grey scale that is known in the art. It is understood that any numerical scale that measures lightness to darkness can be considered a “grey scale”. Moreover, as used herein, “grey scale” is intended to be a linear scale, or one band, or one visual attribute. For example, one “grey scale” visual attribute could be single wavelength or a narrow wavelength to define a specific visual color. Another example of one “grey scale” visual attribute could be a mix of wavelength numerical values averaged for each pixel making up the image, such as a true black, grey or white image from an RGB mixture.

Sensor 46 can take a measurement of the L value of skin surface 18 and/or an image of skin surface 18 and can send it to processor 50 via image capture line 48 for analysis. The image may be analyzed for local L values, background L values, or both. Grey scale conversion can occur within the analytical processing capabilities of processor 50. The comparison of background L to local L to determine the ΔL_{M} occurs within processor 50, which can be a commercially available programmable chip, or other commercially available processing unit.

Processor 50 is generally referred to as a central processing unit (“CPU”). The CPU can be a single programmable chip like those found in consumer electronic devices such as a laptop computer, a cell phone, an electric razor, and the like. The CPU may comprise an Application Specific Integrated Circuit (ASIC), controller, Field Programmable Gate Array (FPGA), integrated circuit, microcontroller, microprocessor, processor, and the like. The CPU may also comprise memory functionality, either internal to the CPU as cache memory, for example Random Access Memory (RAM), Static Random Access Memory (SRAM), and the like, or external to the CPU, for example as Dynamic Random-Access Memory (DRAM), Read Only Memory (ROM), Static RAM, Flash Memory (e.g., Compact Flash or SmartMedia cards), disk drives, Solid State Disk Drives (SSD), or Internet Cloud storage. While it is anticipated that a remote CPU, either tethered to the skincare device 40 or which communicates wirelessly, can be used, a local CPU within the skincare device 40 is exemplified herein.

Images can be taken in sequence or preferably continuously. The sensor (e.g., image sensor or otherwise image capture device) can take images at a speed of at least 4 frames per second, alternatively at least 100 frames per second, alternatively at least 200 frames per second, alternatively at least 600 frames per second.

The CPU can process at a rate of 100 frames per second, alternatively greater than 200 frames per second, and/or alternatively greater than 600 frames per second.

The results of the image analysis, when compared to criteria pre-programmed into processor 50, may result in a desired treatment of skin surface 18. For instance, when the calculated ΔL_{M} exceeds the pre-determined ΔL_{S}, a signal is sent from processor 50 to cartridge 52, via cartridge line 51, to fire one or more nozzles 21 in nozzle array 20 and dispense, or otherwise deposit, the cosmetic ink composition on a user's skin.

Power for cartridge 52, light 44, sensor 46, processor 50, and other mechanical and electrical elements that might be present can be supplied by power element 54 via one or more power lines 55. Power element 54 can be turned off and on, which in turn turns skincare device 40 off and on, via power switch 56 which can be located anywhere on skincare device 40, but is shown here on device cover 58. Power element 54 may include energy storage functionality via a battery, a rechargeable battery, an electrochemical capacitor, a double-layer capacitor, a supercapacitor, a hybrid battery-capacitor system, and/or combinations thereof.

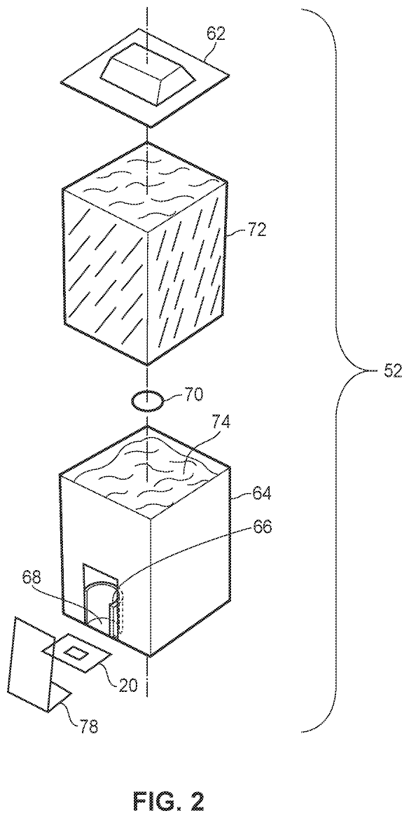

FIG. 2 is an exploded view of a cartridge containing the cosmetic ink composition (e.g., serum) as described for FIG. 1, and in accordance with various aspects disclosed herein. In particular, FIG. 2 shows an exploded view of cartridge 52 comprising cartridge cap 62 and cartridge body 64. Cartridge body 64 can include standpipe 66 which is typically enclosed within cartridge body 64 and defines nozzle outlet 68. Optional filter 70 can help keep excessively large particles, and other debris out of nozzle array 20. Filter 70 and nozzle array 20 can be on opposite sides of nozzle outlet 68. Cosmetic ink composition 74 can be contained within cartridge body 64. Foam core 72 can at least partially fill cartridge 64 and helps to regulate back pressure of cosmetic ink composition 74. Back pressure can be regulated via bladders (not shown) and other methods known to the art. Foam core 72 shown here is just one example of how to help regulate the flow of cosmetic ink composition 74 to standpipe 66 through filter 70 and into nozzle array 20. Connector 78 can provide the electrical power and signal to nozzle array 20. Cosmetic ink composition 74 may be ejected from the cartridge 52 by piezoelectric means, thermal means, mechanical pumping means, or a combination of these.

An exemplary cartridge for use herein can include cartridges described in Patent Application US 2002/0167566, the entirety of which is incorporated by reference herein.

There is no technical difference between an image used for background L values and those used for local L values, the difference is in the analysis of the image. Hence, the images are continually sent to the processor to calculate the L values and ΔL_{M} values. By “sent” it is understood, that preferably at least 4 bits of data per pixel are transferred for each image, and preferably, this 4-bit (or more) packet of data is used in the calculation of each local L value.

It is understood, that the background L can be calculated once in a treatment period and that value can be reused throughout the treatment period. Alternatively, it can be continually recalculated as long as the treatment process goes on. Moreover, there can be pre-programmed triggers to initiate a recalculation of the background L. Also, the background L may be retrieved from the processor memory to be used for the current background L.

When the ΔL_{M} exceeds the predetermined value, the cosmetic ink composition can be deposited onto at least a portion of the skin imperfection. In particular, the cosmetic ink composition can be deposited via an array of nozzles and the local L can be calculated along the length of, and in the firing range of, the array of nozzles. An individual nozzle may be fired to deposit the cosmetic ink composition, or multiple nozzles can be fired at the same time. The number of nozzles fired along the array of nozzles can be adjusted based on the size of the ΔL_{M} and the size of the skin imperfection. Furthermore, the frequency of nozzle firing can be adjusted based on the ΔL_{M}, with more droplets being fired in succession in response to larger ΔL_{M} values.

The skincare device 40 may deposit the cosmetic ink composition in droplets having an average diameter of from about from about 0.1 μm to about 60 μm, alternatively from about 1 μm to about 50 μm, alternatively from about 5 μm to about 40 μm. Preferably, the cosmetic ink composition can be applied to the skin imperfection, or otherwise skin, in a discontinuous pattern of discrete droplets.

The cosmetic ink composition can be printed from a cartridge having a micro-electro-mechanical system (MEMS) that is different from typical consumer printing applications. It is known that the typical chamber height and nozzle plate thicknesses are from about 25 to about 50 μm since typical printing inks have a viscosity of less than about 10 cP. In one aspect, the cartridge can comprise a chamber height and nozzle plate thicknesses of from about 10 to about 20 μm, preferably from about 12 to about 17 μm. Without being limited by theory it is believed that the shorter chamber height and plate thickness can help minimize viscous loss. In addition, most consumer printing applications are optimized for printing at 10 kHz or more, so ink formulas and microfluidics are designed to achieve rapid refill. However, operating the cosmetic ink composition described herein at this frequency range can result in streaming and/or de-priming due to gulping of air.

The cosmetic ink composition can be printed using the following start-up sequence: heating the substrate to about 60° C. for less than about 600 ms, firing the nozzles in a burst of from about 100 to about 500 fires at a frequency of about 300 to about 1000 Hz, and then maintaining the low shear condition with continuous 4 Hz firing. While it is possible the nozzles will start up with different algorithms, it is likely that the cosmetic ink composition would not be transitioned from its viscous at-rest state to a flowing state.

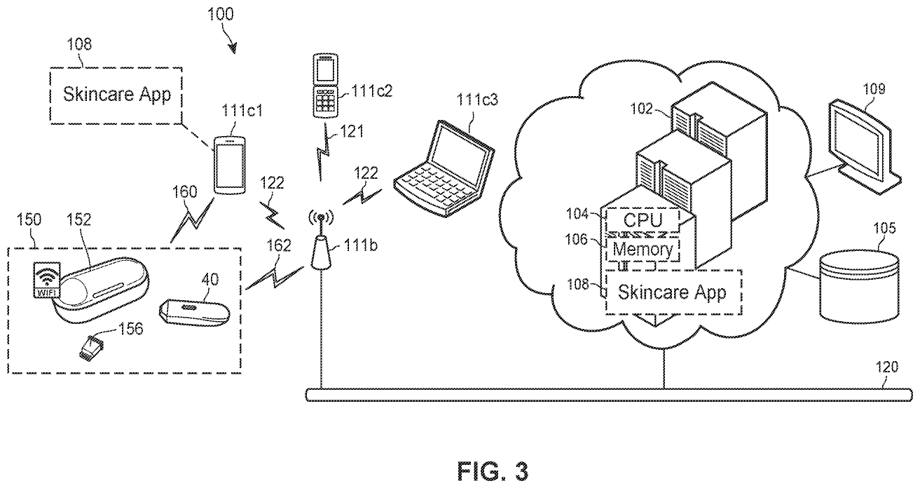

FIG. 3 illustrates an example skincare and facial scanning system 100 configured to provide skincare device connectivity and setting configurations, in accordance with various aspects disclosed herein. A shown in the example of FIG. 3, skincare and facial scanning system 100 comprises a skincare device 40, as described for FIGS. 1 and 2. Skincare device 40 includes a sensor configured to scan human skin and one or more nozzles configured to deposit a cosmetic ink composition (e.g., serum) onto human skin. As exemplified in FIG. 3, skincare device 40 is part of a multi-part skincare device 150 that comprises skincare device 40, cradle 152, and wireless transceiver 156. In the example of FIG. 3, cradle 152 is configured to removably receive or hold skincare device 40. Cradle 152 may charge or store skincare device 40. In some aspects, cradle 152 may provide data connectivity to skincare device 40, where skincare device 40 and transmit and receive data to and from cradle 152 through an electronic and/or digital interface on the skincare device 40 designed to connect to cradle 152 when skincare device 40 is positioned on or within cradle 152.

It should be understood that while FIG. 3 illustrates example multi-part skincare device 150, and that other or different configurations are contemplated herein as well. For example, in one aspect, each of the components of multi-part skincare device 150 (e.g., including the electrical, mechanical, and/or data components of the multi-part skincare device 150) may be combined or integrated into a single device. In another aspect, skincare device 40 and cradle 152 are combined or integrated, but wireless transceiver 52 is configured separately and is adapted to provide communication to and from the combined or integrated device. Other permutations, combinations, and configurations of the components of multi-part skincare device 150 are contemplated herein as well. Generally, as used herein, skincare device 40 can refer to a single device comprising all of the components of multi-part skincare device 150, unless otherwise noted.

In the example of FIG. 3, skincare and facial scanning system 100 includes server(s) 102, which may comprise one or more computer servers. In various aspects server(s) 102 comprise multiple servers, which may comprise multiple, redundant, or replicated servers as part of a server farm. In still further aspects, server(s) 102 may be implemented as cloud-based servers, such as a cloud-based computing platform. For example, server(s) 102 may be any one or more cloud-based platform(s) such as MICROSOFT AZURE, AMAZON AWS, or the like. Server(s) 102 may include a processor 104, which may comprise one or more processors as manufactured or offered by INTEL, AMD, or other server processor or computer processor, or the like.

Server(s) 102 may also comprise computer memory 106. Memory 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memory 106 may store an operating system (OS) (e.g., Microsoft Windows, Linux, UNIX, etc.) capable of facilitating the functionalities, apps, methods, or other software as discussed herein. Memory 106 may also store a skincare application (app) 108, as described herein. In addition, memory 106 may also store machine readable instructions, including any of one or more application(s) (e.g., an imaging application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. It should be appreciated that one or more other applications may be envisioned and that are executed by the processor 104.

The processor 104 may be connected to the memory 106 via a computer bus responsible for transmitting electronic data, data packets, or otherwise electronic signals to and from the processor 104 and memory 106 in order to implement or perform the machine readable instructions, methods, processes, elements or limitations, as illustrated, depicted, or described for the various flowcharts, illustrations, diagrams, figures, and/or other disclosure herein.

Processor 104 may interface with memory 106 via the computer bus to execute an operating system (OS), such as an operating system provided by or based on MICROSOFT, LINUX, or the like. Processor 104 may also interface with the memory 106 via the computer bus to create, read, update, delete, or otherwise access or interact with the data stored in memory 106 and/or the database 105 (e.g., a relational database, such as Oracle, DB2, MySQL, or a NoSQL based database, such as MongoDB). The data stored in memory 106 and/or database 105 may include all or part of any of the data or information described herein, including, for example, user data, including user account data, skincare device data, skincare device usage data, skincare device settings data, or the like, or as otherwise described herein.

Server(s) 102 may further include a communication component configured to communicate (e.g., send and receive) data via one or more external/network port(s) to one or more networks or local terminals, such as computer network 120 and/or terminal 109 (for rendering or visualizing) described herein. In some aspects, server(s) 102 may include a client-server platform technology such as ASP.NET, JAVA J2EE, RUBY ON RAILS, NODE.JS, a web service or online API, responsive for receiving and responding to electronic requests. The server(s) 102 may implement the client-server platform technology that may interact, via the computer bus, with the memories(s) 106 (including the applications(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various aspects, the server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some aspects, computer network 120 may comprise a private network or local area network (LAN). Additionally, or alternatively, computer network 120 may comprise a public network such as the Internet.

Server(s) 102 may further include or implement an operator interface configured to present information to an administrator or operator and/or receive inputs from the administrator or operator. As shown in FIG. 3, an operator interface may provide a display screen (e.g., via terminal 109). Server(s) 102 may also provide I/O components (e.g., ports, capacitive or resistive touch sensitive input panels, keys, buttons, lights, LEDs), which may be directly accessible via, or attached to, server(s) 102 or may be indirectly accessible via or attached to terminal 109. According to some aspects, an administrator or operator may access the server(s) 102 via terminal 109 to review information, make changes, and/or perform other functions.

In some aspects, server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, such as skincare app 108 or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor 104 (e.g., working in connection with the respective operating system in memory 106) to facilitate, implement, or perform the machine readable instructions, methods, processes, elements or limitations, as illustrated, depicted, or described for the various flowcharts, illustrations, diagrams, figures, and/or other disclosure herein. Program code or computing instructions, as describe herein, may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 3, server(s) 102 are communicatively connected, via computer network 120 to one or more user computing devices 111c1-111c3 via base station 111b. In some aspects, base station 111b may comprise cellular base stations, such as cell towers, communicating to the one or more user computing devices 111c1-111c3 via wireless communications 121 based on any one or more of various mobile phone standards, including NMT, GSM, CDMA, UMMTS, LTE, 5G, or the like.

Additionally, or alternatively, base station 111b may comprise wireless routers, wireless switches, or other such wireless connection points communicating to the one or more user computing devices 111c1-111c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 may comprise mobile devices and/or client devices for accessing and/or communications with server(s) 102. In various aspects, user computing devices 111c1-111c3 may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or tablet.

In various aspects, the one or more user computing devices 111c1-111c3 may implement or execute an operating system (OS) or mobile platform such as APPLE iOS and/or Google ANDROID operation system. Any of the one or more user computing devices 111c1-111c3 may comprise one or more processors and/or one or more memories for storing, implementing, or executing computing instructions or code, e.g., a mobile application, as described in various aspects herein. As shown in FIG. 1, skincare app 108 as described herein, or at least portions thereof, may also be stored locally on a memory of a user computing device (e.g., user computing device 111c1).

User computing devices 111c1-111c3 may comprise a wireless transceiver to receive and transmit wireless communications 121 and/or 122 to and from base station 111b. In various aspects, data or information, such as usage data, settings data, and/or firmware updates, may be transmitted via computer network 120 to and from server(s) 102 for analysis, reporting, or otherwise described herein.

Still further, each of the one or more user computer devices 111c1-111c3 may include a display screen for displaying graphics, images, text, mid-section dimension(s), product sizes, data, pixels, features, and/or other such visualizations or information as described herein. In various aspects, graphics, images, text, mid-section dimension(s), product sizes, data, pixels, features, and/or other such visualizations or information may be received from server(s) 102 for display on the display screen of any one or more of user computer devices 111c1-111c3. Additionally, or alternatively, a user computer device may comprise, implement, have access to, render, or otherwise expose, at least in part, an interface or a guided user interface (GUI) for displaying text and/or images on a corresponding display screen.

In some aspects, computing instructions and/or applications executing at the server (e.g., server(s) 102) and/or at a mobile device (e.g., user computing device 111c1) may be communicatively connected for analyzing user-specific data, including skincare device data, usage data, settings data, or other data as describe herein. For example, one or more processors (e.g., processor 104) of server(s) 102 may be communicatively coupled to a mobile device via a computer network (e.g., computer network 120). In some aspects, as shown for FIG. 3, skincare app 108 may comprise a server app portion configured to execute on the one or more processors of the server (e.g., server(s) 102) and a mobile app portion configured to execute on one or more processors of the mobile device (e.g., any of one or more user computing devices 111c1-111c3) and/or other such standalone computing device. In such aspects, the server app portion is configured to communicate with the mobile app portion. The server app portion or the mobile app portion may each be configured to implement, or partially implement, generating, by the skincare app 108 communicatively coupled to the skincare device 40 and/or multi-part skincare device 150, a user-specific electronic analysis based on use of the skincare device on skin of a face portion of the user.

With reference to skincare device 40 and multi-part skincare device 150 of FIG. 3, wireless transceiver 52 may comprise a universal serial bus (USB) dongle configured to transmit wireless signals (e.g., wireless signals 160 and/or 162), including but not limited to wireless signals based on the 802.11 (WIFI) and/or BLUETOOTH standards. Skincare device 40 and/or multi-part skincare device 150 may communicate directly (via wireless signal 162) with base station 111b, e.g., via WIFI or BLUETOOTH and/or via mobile phone standards. Additionally, or alternatively, skincare device 40 and/or multi-part skincare device 150 may communicate (via wireless signal 160) through a user computing device (e.g., user computing device 111c1), where the user computer device is connected to computer network 120 via base station 111b.

Wireless transceiver 52 may be installed in a computing device, such as a personal computer or laptop (e.g., computing device 111c3), router, universal serial bus (USB) port, or other port or system to communicatively couple cradle 152 and/or skincare device 40 to a computer network (e.g., such as computer network 120). For example, wireless transceiver 52 may be configured for installation into a laptop (e.g., user computing device 111c3) or other computing device to provide connectivity to computer network 120. In other aspects, cradle 152 may communicate directly with a computing device (e.g., user computing device 111c1) having or already configured with a BLUETOOTH and/or WIFI transceiver, and without the need for wireless transceiver 52.

In addition, cradle 152 and skincare device 40 may be communicatively coupled, e.g., wired or wirelessly (e.g., via wireless signals based on the 802.11 (WIFI) and/or BLUETOOTH standards) for the transmission of data. Data transmission, whether to or from skincare device 40 to cradle 152, and/or, more generally, over computer network 120 (e.g., to or from either of cradle 152 and/or skincare device 40), may comprise, by way of non-limiting example, data transmission regarding use, operation, settings, firmware updates, feedback, analysis, reports, or other such data or otherwise information as described herein.

In various aspects, a skincare application (app) 108 is configured to communicate with a multi-part skincare device 150, or any of the components thereof (e.g., skincare device 40, cradle 152, and/or wireless transceiver 52). Skincare app 108 comprises computing instructions such as, by way of non-limiting example, JavaScript, HTML, Java, python, C, C#, GO, computing instructions or the like. The instructions may be stored on a tangible, non-transitory computer-readable medium (e.g., RAM or ROM memory) and may be configured to provide skincare device connectivity and setting configurations as described herein. In some aspects, the skincare app may be coded or configured as a web application operating at a web site, server (e.g., server 102, as described herein), such as a cloud server or other server. In other aspects, skincare app 108 may comprise a mobile app, such as a native mobile app (e.g., configured for a specific operating system such as APPLE IOS or GOOGLE ANDROID), configured for operation on a mobile device (e.g., an IPHONE device and/or an ANDROID device).

Skincare app 108 may be configured for communication with skincare device 40 or otherwise multi-part skincare device 150, or any of the components thereof. Skincare app 108 is configured for operation on one or more processors, including one or more processors of a mobile device (e.g., an IPHONE device), one or more processors a server (e.g., server 102, as described herein), and/or the skincare device itself. It is to be understood that the one or more processors may be remotely located to one another such that skincare app 108 is a distributed application having client-side code or instructions designed to communicate with server-side code or instructions. In such aspects, a client-server technology such as Active Server Pages (ASP), Java Server Pages (JSP), EXPRESS, ANGULAR, NODE.JS, or the like may be used to implement or otherwise configure skincare app 108. In still other aspects, skincare app 108 may be configured to run locally on a single device, e.g., on a mobile app of a mobile device (e.g., an IPHONE device) in communication with a multi-part skincare device 150, or any of the components thereof (e.g., skincare device 40, cradle 152, and/or wireless transceiver 52).

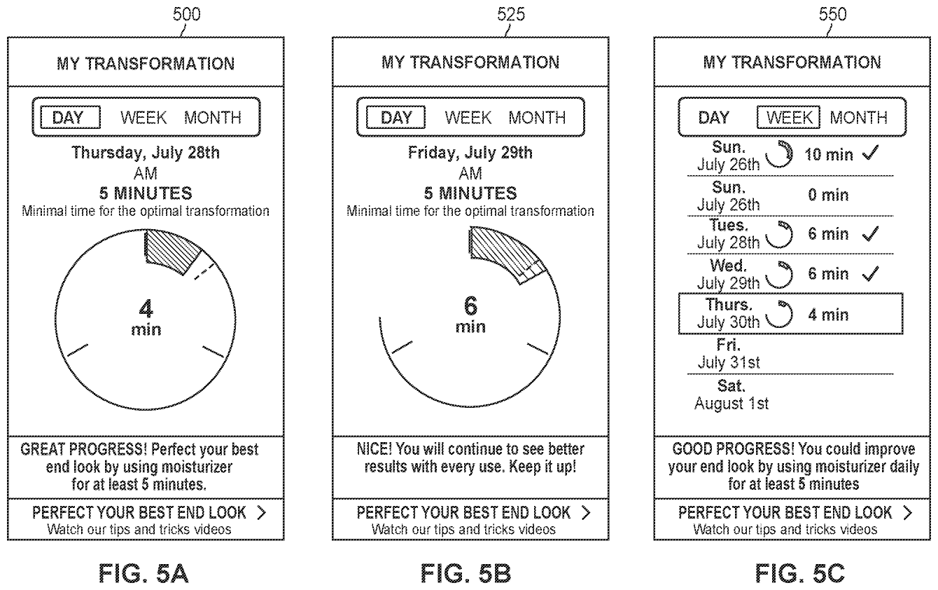

The computing instructions of skincare app 108, when executed by one or more processors, may cause the one or more processors to generate a user-specific electronic analysis based on use of the skincare device on skin of a face portion of the user. The user-specific electronic analysis may comprise data, insights, recommendations, and/or reports specific to the user, specific to the user's skincare device, and/or specific to the user's use of the skincare device, e.g., such as use overtime. Examples of such data, insights, recommendations, and/or reports specific to the user, specific to the user's skincare device, and/or specific to the user's use of the skincare device are described herein, by way of non-limiting example, for FIGS. 5A-5C and/or 6A-6C, or as otherwise described herein.

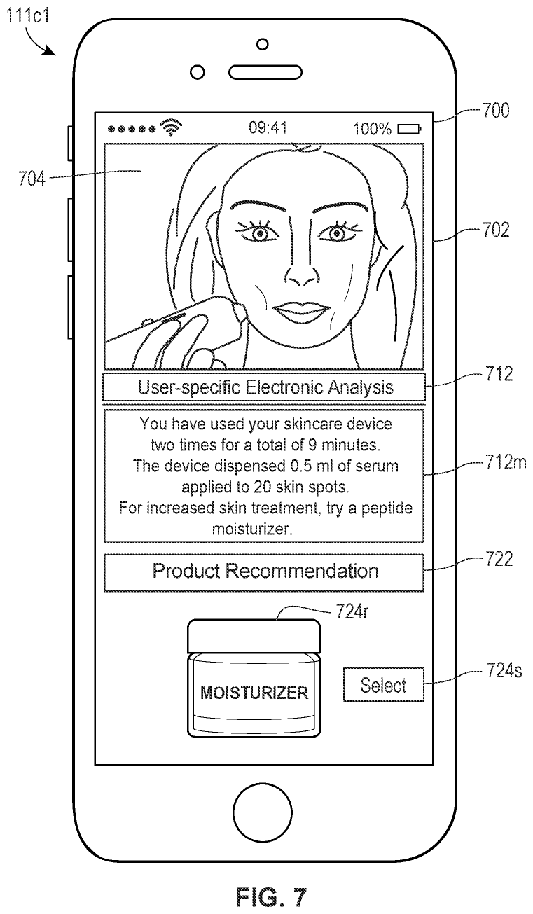

In various aspects, at least a portion of the user-specific electronic analysis, for example as shown for any of the FIGs or as otherwise described herein, is configured for display on a graphic user interface (GUI). In some aspects, the GUI may be integrated as part of a graphic user interface as displayed on a display screen of the skincare device itself. In other aspects, the GUI may be configured to operate or execute a mobile device, such as via a mobile application on an IPHONE device or similar device. An example GUI is shown and described herein, by way of non-limiting example, for FIG. 7, or as otherwise described herein.

With reference to FIG. 3, example skincare and facial scanning system 100 provides provide skincare device connectivity and setting configurations as part of a feedback loop that allows a skincare device (e.g., skincare device 40) to be connected to computer network 120 and/or server(s) 102. In various aspects, a connection may be a persistent connection maintaining connectivity between the skincare device 40 and the skincare app 108 for a plurality of uses of the skincare device 40. The persistent connection can provide an always-on or almost always-on connection that is maintained across two or more uses of skincare device 40. That is, a skincare device (e.g., skincare device 40) can remain connected or otherwise can maintain a connection t to establish a feedback loop (e.g., an always-on feedback loop) that provides usage insights and offers incremental future new benefits driven by pushing new software based features or fixes to the skincare device based on user-specific usage, skin type, and/or preferences.

Connectivity may be established and/or maintained by skincare device 40 uniquely identifying itself across computer network 120 and/or at least to server(s) 102. For example, skincare device 40 may comprise one or more identification certificates (such as one or more digital certificates or security certificates) stored on a memory of the skincare device. In such aspects, the identification certificate may be configured to uniquely identify the skincare device to the skincare app upon a connection between skincare device 40 and the skincare app 108. For example, the identification certificate may comprise a public key, secure key, or otherwise identification key (e.g., unique alphanumeric value) stored on skincare device 40. Such identification certificate, or counterpart thereto (e.g., such as a private key) may be stored on the skincare app 108 and/or server(s) 102 such that the skincare app 108 and/or server(s) 102 may recognize the skincare device 40 upon receipt of the identification certificate or portion thereof (e.g., a public key). The skincare app 108 and/or server(s) 102 may transmit the identification certificate or portion thereof across the network to the skincare device 40 in response and/or may use the identification certificate or portion thereof to establish a connection with that specific skincare device 40. As described herein, the connection may comprise a WIFI connection and/or BLUETOOTH connection. Additionally, or alternatively, the connection may comprise mobile standard connection, otherwise an internet connection, e.g., connecting and identifying skincare device 40 across computer network 120 to server(s) 102.

In some aspects, the skincare app 108, when executed by the one or more processors (e.g., of user computing device 111c1 and/or server(s) 102), cause the one or more processors to pair the identification certificate of the skincare device to a digital account of the user. Pairing the identification certificate to the digital account of the user allows the skincare device 40 to be uniquely identified across computer network 120 and/or at least to server(s) 102. Such pairing also allows for onboarding the skincare device to allow for image and device tracking. In this way, in various aspects, the identification certificate is assigned to an account of the user and is used to identify the skincare device 40 to the skincare app 108 and to provision the device to establish a connection (e.g., an always-on connection) via the consumer's home network (e.g., via base station 111b) or through the user's mobile network to the skincare app (directly) and/or through the server(s) 102, which, in some examples, can be an internal secured cloud/server, such as Amazon AWS, Microsoft AZURE, or the like, or as otherwise described herein.

By way of example, in one aspect, to setup a new skincare device 40 a user may onboard or connect her the skincare device 40 via skincare app 108 (e.g., a website, mobile application, or point of input). In this process, the user can establish her identity (e.g., via an identification certificate) and the purchased OPTE device together via the skincare app 108. Skincare device 40 may contain one or more identification certificates (e.g., digital certificates) that enable the skincare device 40 to connect only to the secure server(s) 102. Skincare device 40, in communication with server(s) 102 and identified by identification certificate(s), establish a connection (e.g., an “always on” connection) over Bluetooth or WIFI such that the skincare device 40 can communicate its data and/or information to the server(s) 102, which may then communicate analysis, reports, insights, or otherwise information the user based on usage of or information about skincare device 40. For example, once skincare device 40 is successfully connected, the user will be able to view a series of data and insights from the connected skincare device 40 for the life of the product as long it is within the sufficient range of a wireless and/or wired connection, as described herein.

In various aspects, usage or device settings data defining the use or operation of the skincare device is associated with the digital account of the user. For example, in various aspects, usage or device settings data may comprise at least one of: (1) an amount of the cosmetic ink composition deposited on the skin of the user; (2) a number of scans of the skin of the user; or (3) a time or a duration of usage of the skincare device by the user. Other examples of usage and/or device settings, or otherwise of benefits a connected skincare device 40, include tracking of user-specific usage activity, tracking and communication of the condition of the skincare device 40 and the cartridge or serum, and the provision to both the user and the manufacturer of the skincare device 40 data and information of the skincare device 40 which allows for the ability to maintain, upgrade, and/or create new capabilities that can be digitally pushed to the user to deliver new features via software driven functionality over the life of the skincare device 40.

For example, in various aspects, the computing instructions of the skincare app 108, when executed by the one or more processors, may cause the one or more processors to upgrade a firmware of the skincare device based on at least one of: the condition status of the skincare device or the use of the skincare device on skin of the face portion of the user. A firmware update may cause a new or different operation of the skincare device. A new or different operation may comprise, by way of non-limiting example, to cause skincare device 40 to apply serum in a new or different pattern, etc., for a new or different type of skin scanned by the sensor of the skincare device 40. Still further, in additional non-limiting examples, a new or different operation, e.g., as introduced by a firmware update, can include causing the skincare device 40 to print more or less cosmetic product; personalizing the skincare device to the user (e.g., by different scan patterns and/or serum application and/or by changing or adding audio and/or visual features to skincare device 40). For example, a firmware update may adjust a one or more settings of the skincare device 40 that can range from how much serum it sprays or deposits to how little is sprays or deposits, how much it clicks and/or how little it clicks (e.g., audible clicks when operating), how many drops are printed in one spray, etc. Once the skincare device 40 is adjusted with the firmware update, a user may operate the skincare device 40 using the adjusted conditions for a more personalized or customized scanning and/or serum depositing (e.g., cosmetic ink composition 74 application) results.

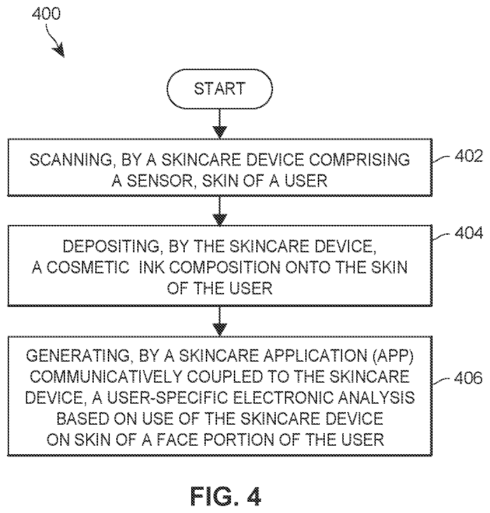

FIG. 4 illustrates an example skincare and facial scanning method 400 for providing skincare device connectivity and setting configurations, in accordance with various aspects disclosed herein. At block 402, skincare and facial scanning method 400 comprises scanning, by a skincare device comprising a sensor (e.g., sensor 46), skin (e.g., skin surface 18) of a user. The skincare device may be, by way of non-limiting example, skincare device 40 and/or multi-part skincare device 150 as described herein.

At block 404, skincare and facial scanning method 400 comprises depositing, by the skincare device (e.g., skincare device 40 and/or multi-part skincare device 150), a cosmetic ink composition onto the skin of the user. The cosmetic ink composition may be or comprise a serum such as cosmetic ink composition 74 as described herein.

At block 406, skincare and facial scanning method 400 comprises generating, by a skincare application (app) (e.g., skincare app 108) communicatively coupled to the skincare device, a user-specific electronic analysis based on use of the skincare device (e.g., skincare device 40) on skin of a face portion of the user. The user-specific electronic analysis may comprise a report, data, insight, recommendations and/or other information specific to the user, the user's skin, and/or the skincare device 40 and/or otherwise multi-part skincare device 150. In various aspects, at least a portion of the user-specific electronic analysis is configured for display on a graphic user interface (GUI), for example as described herein for FIG. 7.

FIGS. 5A-5C illustrate example GUIs, each depicting a user-specific electronic analysis as described herein. FIG. 5A illustrates an example graphic user interface 500 depicting a user-specific electronic analysis based on use of the skincare device as describe for FIG. 1, in accordance with various aspects disclosed herein. In the example of FIG. 5A, GUI 500 depicts data of a user captured at a first time, e.g., on Thursday, July 28^{th} in the morning (“AM”), where the user used or otherwise operated the skincare device 40 for 4 minutes. Such data may have been collected by skincare device 40 and transmitted to (and optionally stored on) server(s) 102 across computer network 120 as described herein, and used for generating the user-specific electronic analysis. The user-specific electronic analysis also includes a recommendation or insight for the user, comprising information and encouraging the user that 5 minutes is the minimal time for optimal skin transformation.

FIG. 5B illustrates a further example graphic user interface 525 depicting a user-specific electronic analysis based on use of the skincare device as describe for FIG. 1, in accordance with various aspects disclosed herein. In the example of FIG. 5B, GUI 525 depicts data of a user captured at a second time, e.g., on Friday, July 29^{th} in the morning (“AM”), where the user used or otherwise operated the skincare device 40 for 5 minutes. Such data may have been collected by skincare device 40 and transmitted to (and optionally stored on) server(s) 102 across computer network 120 as described herein, and used for generating the user-specific electronic analysis. The user-specific electronic analysis also includes an insight or report for the user, comprising information and identifying that the user successfully met the target of operating the skincare device 40 for 5 minutes for optimal skin transformation.

FIG. 5C illustrates an additional example graphic user interface 550 depicting a user-specific electronic analysis based on use of the skincare device as describe for FIG. 1, in accordance with various aspects disclosed herein. The example of FIG. 5C depicts additional data of the user as captured on a weekly basis (e.g., for a specific week), where the user used or operated the device at various times (e.g., 10 minutes, 0 minutes, 6 minutes, and 4 minutes) for the days of July 26h to August 1^{st}. The user-specific electronic analysis also includes an insight or report to the user, comprising information and identifying that the user is successfully operating the skincare device 40, within a weekly period, for optimal skin transformation and/or appearance. Although FIGS. 5A-5C show daily and weekly data, it is to be understood that other data for other time periods may also be shown. For example, e.g., monthly, yearly, or custom range data may also be determined and/or depicted.

In various aspects, skincare and facial scanning system 100 may comprise tracking of a user's skincare device usage and/or activity. For example, FIGS. 5A-5C illustrate tracking of a user's skincare device usage and/or activity. In such examples, usage data defining use of the skincare device (e.g., skincare device 40) may be captured over time including at a first time and a second time as illustrated for FIGS. 5A and 5B. Such data may be captured after a device connection is made, e.g., with server(s) 102 across computer network 120, as described herein. In such aspects, a user-specific electronic analysis (e.g., report, insight, and/or recommendation as illustrated for FIGS. 5A-5C) may be based on tracking usage data, as a user operated skincare device 40, as captured at the first time and the second time. Such data may be collected over various uses or operations of the skincare device 40, which such data may be transmitted to and/or stored at server(s) 102.

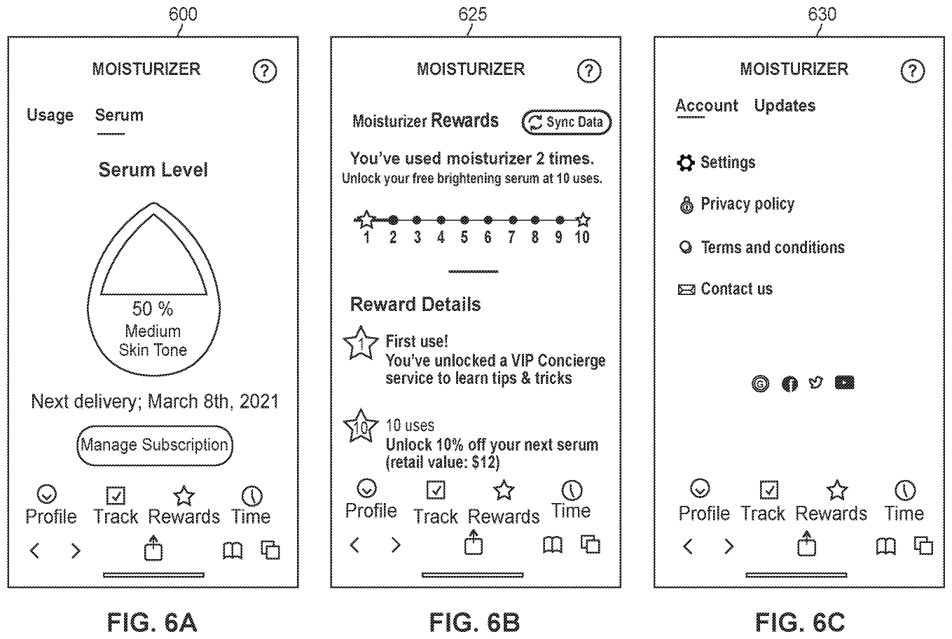

FIG. 6A illustrates an example graphic user interface 600 depicting a recommendation for a product, in accordance with various aspects disclosed herein. In the example of FIG. 6A, additional serum (e.g., cosmetic ink composition 74) is recommended. In such aspects, skincare and facial scanning system 100, by communication from skincare device 40 to server(s) 102 and/or by skincare device 40 alone, may track and/or display the condition of the skincare device 40 and/or the cartridge or serum contained in the cartridge. For example, in various aspects, the computing instructions of the skincare app 108, when executed by the one or more processors (e.g., one or more processors of user computing device 111c1 and/or server(s) 102), may cause the one or more processors to obtain a condition status of the skincare device 40, and/or its cartridge 52, and generate device-specific data for the skincare device. The device-specific data may include cartridge data (e.g., amount or level of cosmetic ink or otherwise serum remaining), device settings, and/or calibration settings of skincare device 40.

FIG. 6B illustrates an example graphic user interface 625 depicting usage data defining use of the skincare device 40 as captured over time, in accordance with various aspects disclosed herein. For example, as shown for GUI 625, a user's usage data (e.g., use of two times) is tracked. Such usage data may have been capture over time, for example, as described for FIGS. 5A-5C as described herein. The user-specific electronic analysis as shown for FIG. 6B includes reward(s) (e.g., information such as how to use the skincare device 40 and/or discounts or otherwise offers on additional serum, e.g., cosmetic ink composition 74) based on the user's usage or operation of the skincare device 40 as tracked and/or captured overtime by skincare and facial scanning system 100.

FIG. 6C illustrates an example graphic user interface 650 depicting a usage or device settings screen for editing usage or device settings data of the skincare device, in accordance with various aspects disclosed herein. GUI 650 may be used to edit and/or update usage and/or device settings of skincare device 40 and may be accessible via skincare app 108. For example, in various aspects, the computing instructions of the skincare app 108, when executed by the one or more processors (e.g., one or more processors of user computing device 111c1 and/or server(s) 102), may cause the one or more processors to: receive a device setting configurable by the user, and adjust an operation of the skincare device. In various aspects, adjusting operation of the skincare device (e.g., skincare device 40) may comprise at least one of: printing a different quantity of cosmetic ink composition; providing information about the skincare device in a display, alerting or modifying a display setting of the skincare device; or altering an output sound (e.g., click) of the skincare device. For example, a user may adjust, via the GUI settings, sounds (e.g. the clicks) that skincare device 40 makes as it deposits serum on skin (e.g., skin surface 18). As an additional, example information as provided in a display (e.g., a display of the skincare device 40 or a user computing device) may comprise information that may be different or altered information, e.g., including operation of the skincare device as the skincare device moves over the surface of the user's skin (e.g., skin surface 18).

In some aspects, device and/or usage settings may be predefined. For example, a device setting may comprise a predefined device setting causing the skincare device to operate in a predefined device mode. A predefined device setting comprises a selectable, predetermined setting that a user can select. Such features may be customized by the user based on the user's preferences. For example, one such predefined device setting may include “more precise, less coverage,” that can cause skincare device 40 to operate for application session when the user does not need or want a full facial application of makeup. Other examples of predefined device settings include an “add higher coverage” predefined device setting or a “faster coverage” predefined device setting for operating skincare device 40 when the user wants to apply serum with speed or where the user needs to cover her spots more thoroughly, respectively. In any event, once a user selects a predefined device setting, such predefined device setting may be sent to the skincare app 108 and/or server(s) 102. The skincare app 108 and/or server(s) 102 may then push or otherwise transmit a firmware update to the skincare device 40 to cause the skincare device 40 to operate in the manner as specified by the predefined device setting.

Other settings, configurations, updates, etc. may be pushed to skincare device 40 or otherwise multi-part skincare device 150 in the same or similar manner to update or change the operation of skincare device 40 and/or multi-part skincare device 150. Additionally, or alternatively, in some aspects such updates, such as firmware updates, may be stored on a memory of skincare device 40 (e.g., preloaded) where the firmware update may happen without communication with server(s) 102, and where the firmware update happens wholly on the skincare device 40 based on a user's selection.

FIG. 7 illustrates an example graphic user interface 702 as rendered on a display screen 700 of a user computing device (e.g., user computing device 111c1) in accordance with various aspects disclosed herein. For example, as shown in the example of FIG. 7, user interface 702 may be implemented or rendered via an application (app executing on user computing device 111c1). For example, as shown in the example of FIG. 7, user interface 702 may be implemented or rendered via a native app executing on user computing device 111c1. In the example of FIG. 7, user computing device 111c1 is a user computer device as described for FIG. 1, e.g., where 111c1 is illustrated as an APPLE iPhone that implements the APPLE iOS operating system and that has display screen 700. User computing device 111c1 may execute one or more native applications (apps) on its operating system, including, for example, an imaging app (e.g., skincare app 108) as described herein. Such native apps may be implemented or coded (e.g., as computing instructions) in a computing language (e.g., SWIFT) executable by the user computing device operating system (e.g., APPLE iOS) by the processor of user computing device 111c1. In various aspects, the imaging app (e.g., an skincare app 108) executing on a mobile devices, such as user computing device 111c1, may be referred to as an “skincare” app or “advisor” app (e.g., skincare app 108), designed to display or render a user-specific electronic analysis based on use of the skincare device on skin of a face portion of the user, or, more generally GUIs, such as described herein with respect to any one or more of FIGS. 1-4, 5A-5C, and/or 6A-6C.

Additionally, or alternatively, user interface 702 may be implemented or rendered via a web interface, such as via a web browser application, e.g., Safari and/or Google Chrome app(s), or other such web browser or the like.

In the example of FIG. 7, GUI 702 displays image 704. Image 704 may comprise an image of the user as uploaded by skincare app 108 to user computing device 111c1 and/or server(s) 102. In other aspects, image 704 may comprise an image of another individual applying a product (e.g., a recommended product), such as the serum, or such as a specific type or quantity of serum, etc., as deposited by skincare device 40.

A recommendation, such as product recommendation 722, may be transmitted, via the computer network (e.g., from a server 102 and/or one or more processors) to user computing device 111c1, for rendering on display screen 700.

In various aspects, skincare app 108, as executing on user computing device 111c1, may cause the one or more processors to: generation a recommendation for a product based on at least one of: the condition status of the skincare device, the use of the skincare device on skin of the face portion of the user, the use of the skincare device on skin of a body portion of the user. The recommendation may be part of, or based on, a user-specific electronic analysis as described herein. Additionally, or alternatively, the recommendation may be further based on user data as provided by the user to the skincare app. For example, in the example of FIG. 7, a product recommendation is made based on a user-specific electronic analysis 712 involving analysis of use of the skincare device on skin of the user. GUI 702 informs the user, via message 712m, of usage of the device, serum levels used, number of skin spots detected, and provides a product recommendation.

More generally, product recommendations, may include, by way of non-limiting example, a recommendation for an optimal product to use for or with skincare device 40 to cause a desired improvement or visual appearance for the user's skin.

In the example of FIG. 7, user interface 702 renders or provides a recommended product (e.g., manufactured product 724r, which may comprise a skin moisturizer) as determined by the skincare app 108 and the related analysis of skin surface 18 as scanned by sensor 46. In the example of FIG. 7, the recommendation may be based on user-specific electronic analysis and/or by the user's input, where a user may have indicated a desire to add a moisturizing effect or look to the user's appearance.

User interface 702 may further include a selectable user interface (UI) button 724s to allow the user to select for purchase or shipment the corresponding product (e.g., manufactured product 724r). In some aspects, selection of selectable UI button 724s may cause the recommended product(s) to be shipped to the user and/or may notify a third party that the individual is interested in the product(s). In such aspects, the product may be packaged and shipped to the user.

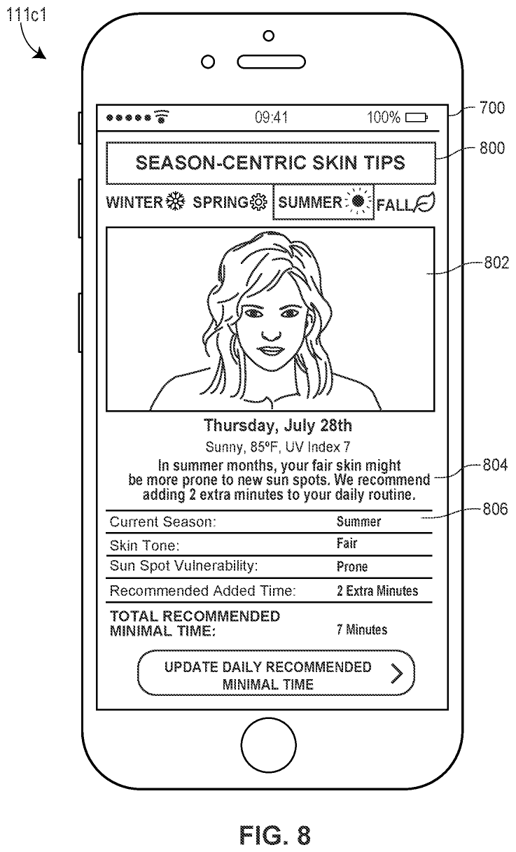

FIG. 8 illustrates a further example graphic user interface 800 as rendered on a display screen of a user computing device in accordance with various aspects disclosed herein. In the example of FIG. 8, a user 802 has usage data is analyzed and displayed with weather data 806 comprising current or expected weather of a location that the user is currently or expected to be located. Such local weather data may be downloaded from a third-party server and/or otherwise stored on server(s) 102 for analysis by skincare app 108, or otherwise user computing device 111c1 and/or server(s) 102. As shown for FIG. 8, when analyzed together, the weather data 806 and the user's specific usage data allows skincare app 108 provide an operation recommendation 804 of skincare device 40 on the user's skin based on the weather data.

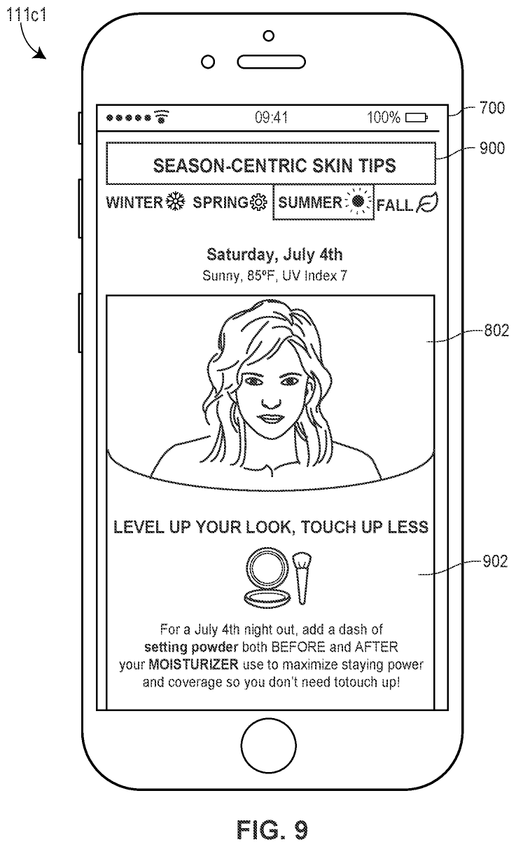

FIG. 9 illustrates a further example graphic user interface 900 as rendered on a display screen of a user computing device in accordance with various aspects disclosed herein. In the example of FIG. 9, a product recommendation 902 is recommended to the user 802 based on the weather data of FIG. 8 and the user's user specific data as described herein. The product recommendation can be for a product for a specific date and/or expected weather or temperature, as determined from weather data, or as described for FIG. 7 or as otherwise described herein.

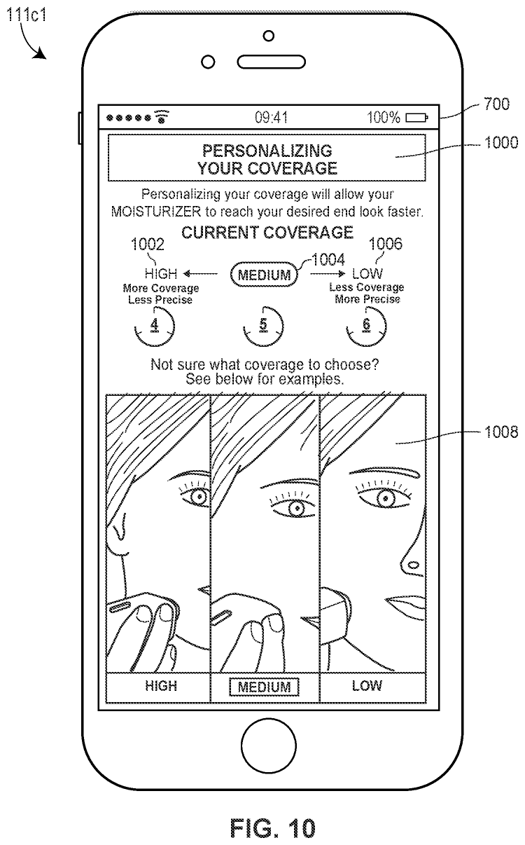

FIG. 10 illustrates a further example graphic user interface 1000 as rendered on a display screen of a user computing device in accordance with various aspects disclosed herein. The example of FIG. 10 shows an email GUI 1000 where the user may select a predefined setting and/or usage setting as described herein. In the example of FIG. 10, the user may select from high coverage 1002, medium coverage 1004, and low coverage 1006 as settings for skincare device 40 to cause skincare device 40 to apply different amounts of serum to the user's skin in order to give the user a desired look or appearance based on the user's selection. Example images 1008 of application of serum, upon or after selection of the settings (e.g., 1002, 1004, and 1006) may be depicted as shown for FIG. 10.

### Aspects of the Disclosure

The following aspects are provided as examples in accordance with the disclosure herein and are not intended to limit the scope of the disclosure.

1. A skincare and facial scanning system configured to provide skincare device connectivity and setting configurations, the skincare and facial scanning system comprising: a skincare device comprising a sensor configured to scan human skin and one or more nozzles configured to deposit a cosmetic ink composition onto human skin; and a skincare application (app) comprising computing instructions and communicatively coupled to the skincare device, wherein the computing instructions of the skincare app, when executed by one or more processors, cause the one or more processors to: generate a user-specific electronic analysis based on use of the skincare device on skin of a face portion of the user, wherein at least a portion of the user-specific electronic analysis is configured for display on a graphic user interface.

2. The skincare and facial scanning system of aspect 1, wherein the skincare device further comprises an identification certificate stored on a memory of the skincare device, and wherein the identification certificate is configured to uniquely identify the skincare device to the skincare app upon a connection between the skincare device and the skincare app.

3. The skincare and facial scanning system of aspect 2, wherein the connection is a persistent connection maintaining connectivity between the skincare device and the skincare app for a plurality of uses of the skincare device.

4. The skincare and facial scanning system of aspect 2, wherein the computing instructions of the skincare app, when executed by the one or more processors, cause the one or more processors to: pair the identification certificate of the skincare device to a digital account of the user, wherein usage or device settings data defining the use or operation of the skincare device is associated with the digital account of the user.

5. The skincare and facial scanning system any one of aspects 1-4, wherein usage data defining use of the skincare device is captured over time including at a first time and a second time, and wherein the user-specific electronic analysis is based on tracking usage data captured at the first time and the second time.

6. The skincare and facial scanning system of aspect 4, wherein the usage or device settings data comprises at least one of: (1) an amount of the cosmetic ink composition deposited on the skin of the user; (2) a number of scans of the skin of the user; or (3) a time or a duration of usage of the skincare device by the user.

7. The skincare and facial scanning system any one of aspects 1-7, wherein the computing instructions of the skincare app, when executed by the one or more processors, cause the one or more processors to: obtain a condition status of the skincare device, and generate device-specific data for the skincare device.

8. The skincare and facial scanning system of aspect 7, wherein the computing instructions of the skincare app, when executed by the one or more processors, cause the one or more processors to: generation a recommendation for a product based on at least one of: the condition status of the skincare device, the use of the skincare device on skin of the face portion of the user, the use of the skincare device on skin of a body portion of the user.

9. The skincare and facial scanning system of aspect 8, wherein the recommendation is further based on user data as provided by the user to the skincare app.

10. The skincare and facial scanning system of aspect 7, wherein the computing instructions of the skincare app, when executed by the one or more processors, cause the one or more processors to: upgrade a firmware of the skincare device based on at least one of: the condition status of the skincare device or the use of the skincare device on skin of the face portion of the user, wherein the firmware causes a new or different operation of the skincare device.

11. The skincare and facial scanning system any one of aspects 1-10, wherein the computing instructions of the skincare app, when executed by the one or more processors, cause the one or more processors to: receive a device setting configurable by the user, and adjust an operation of the skincare device.

12. The skincare and facial scanning system of aspect 11, wherein adjusting the operation of the skincare device comprises at least one of: printing a different quantity of cosmetic ink composition; providing information about the skincare device in a display, alerting or modifying a display setting of the skincare device; or altering an output sound of the skincare device.

13. The skincare and facial scanning system any one of aspects 1-12, wherein the device setting is a predefined device setting causing the skincare device to operate in a predefined device mode.

14. A skincare and facial scanning method for providing skincare device connectivity and setting configurations, the skincare and facial scanning method comprising: scanning, by a skincare device comprising a sensor, skin of a user; depositing, by the skincare device, a cosmetic ink composition onto the skin of the user; and generating, by a skincare application (app) communicatively coupled to the skincare device, a user-specific electronic analysis based on use of the skincare device on skin of a face portion of the user, wherein at least a portion of the user-specific electronic analysis is configured for display on a graphic user interface.

15. The skincare and facial scanning method of aspect 14, wherein the skincare device further comprises an identification certificate stored on a memory of the skincare device, and wherein the identification certificate is configured to uniquely identify the skincare device to the skincare app upon a connection between the skincare device and the skincare app.

16. The skincare and facial scanning method of aspect 15, wherein the connection is a persistent connection maintaining connectivity between the skincare device and the skincare app for a plurality of uses of the skincare device.

17. The skincare and facial scanning method of aspect 15, further comprising: pairing the identification certificate of the skincare device to a digital account of the user, wherein usage or device settings data defining the use or operation of the skincare device is associated with the digital account of the user.

18. The skincare and facial scanning method any one of aspects 14-17, wherein usage data defining use of the skincare device is captured over time including at a first time and a second time, and wherein the user-specific electronic analysis is based on tracking usage data captured at the first time and the second time.

19. The skincare and facial scanning method of aspect 17, wherein the usage or device settings data comprises at least one of: (1) an amount of the cosmetic ink composition deposited on the skin of the user; (2) a number of scans of the skin of the user; or (3) a time or a duration of usage of the skincare device by the user.

20. A tangible, non-transitory computer-readable medium storing instructions for providing skincare device connectivity and setting configurations, that when executed by one or more processors cause the one or more processors to: scan, by a skincare device comprising a sensor, skin of a user; deposit, by the skincare device, a cosmetic ink composition onto the skin of the user; and generate a user-specific electronic analysis based on use of the skincare device on skin of a face portion of the user, wherein at least a portion of the user-specific electronic analysis is configured for display on a graphic user interface.

### ADDITIONAL CONSIDERATIONS

Although the disclosure herein sets forth a detailed description of numerous different aspects, it should be understood that the legal scope of the description is defined by the words of the claims set forth at the end of this patent and equivalents. The detailed description is to be construed as exemplary only and does not describe every possible aspect since describing every possible aspect would be impractical. Numerous alternative aspects may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain aspects are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example aspects, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example aspects, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example aspects, the processor or processors may be located in a single location, while in other aspects the processors may be distributed across a number of locations.

The performance of certain of the operations may be distributed among the one or more processors, not only residing within a single machine, but deployed across a number of machines. In some example aspects, the one or more processors or processor-implemented modules may be located in a single geographic location (e.g., within a home environment, an office environment, or a server farm). In other aspects, the one or more processors or processor-implemented modules may be distributed across a number of geographic locations.

This detailed description is to be construed as exemplary only and does not describe every possible aspect, as describing every possible aspect would be impractical, if not impossible. A person of ordinary skill in the art may implement numerous alternate aspects, using either current technology or technology developed after the filing date of this application.

Those of ordinary skill in the art will recognize that a wide variety of modifications, alterations, and combinations can be made with respect to the above described aspects without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skincare and facial scanning system configured to provide skincare device connectivity and setting configurations, the skincare and facial scanning system comprising:
a skincare device comprising a sensor configured to scan human skin and one or more nozzles configured to deposit a cosmetic ink composition onto human skin; and
a skincare application (app) comprising computing instructions and communicatively coupled to the skincare device,
wherein the computing instructions of the skincare app, when executed by one or more processors, cause the one or more processors to:
generate a user-specific electronic analysis based on use of the skincare device on skin of a face portion of the user,
wherein at least a portion of the user-specific electronic analysis is configured for display on a graphic user interface.

2. The skincare and facial scanning system of claim 1,
wherein the skincare device further comprises an identification certificate stored on a memory of the skincare device, and
wherein the identification certificate is configured to uniquely identify the skincare device to the skincare app upon a connection between the skincare device and the skincare app.

3. The skincare and facial scanning system of claim 2, wherein the connection is a persistent connection maintaining connectivity between the skincare device and the skincare app for a plurality of uses of the skincare device.

4. The skincare and facial scanning system of claim 2, wherein the computing instructions of the skincare app, when executed by the one or more processors, cause the one or more processors to:
pair the identification certificate of the skincare device to a digital account of the user,
wherein usage or device settings data defining the use or operation of the skincare device is associated with the digital account of the user.

5. The skincare and facial scanning system of claim 1,
wherein usage data defining use of the skincare device is captured over time including at a first time and a second time, and
wherein the user-specific electronic analysis is based on tracking usage data captured at the first time and the second time.

6. The skincare and facial scanning system of claim 4, wherein the usage or device settings data comprises at least one of: (1) an amount of the cosmetic ink composition deposited on the skin of the user; (2) a number of scans of the skin of the user; or (3) a time or a duration of usage of the skincare device by the user.

7. The skincare and facial scanning system of claim 1, wherein the computing instructions of the skincare app, when executed by the one or more processors, cause the one or more processors to:
obtain a condition status of the skincare device, and
generate device-specific data for the skincare device.

8. The skincare and facial scanning system of claim 7, wherein the computing instructions of the skincare app, when executed by the one or more processors, cause the one or more processors to:
generation a recommendation for a product based on at least one of: the condition status of the skincare device, the use of the skincare device on skin of the face portion of the user, the use of the skincare device on skin of a body portion of the user.

9. The skincare and facial scanning system of claim 8, wherein the recommendation is further based on user data as provided by the user to the skincare app.

10. The skincare and facial scanning system of claim 7, wherein the computing instructions of the skincare app, when executed by the one or more processors, cause the one or more processors to:
upgrade a firmware of the skincare device based on at least one of: the condition status of the skincare device or the use of the skincare device on skin of the face portion of the user,
wherein the firmware causes a new or different operation of the skincare device.

11. The skincare and facial scanning system of claim 1, wherein the computing instructions of the skincare app, when executed by the one or more processors, cause the one or more processors to:
receive a device setting configurable by the user, and
adjust an operation of the skincare device.

12. The skincare and facial scanning system of claim 11, wherein adjusting the operation of the skincare device comprises at least one of: printing a different quantity of cosmetic ink composition; providing information about the skincare device in a display, alerting or modifying a display setting of the skincare device; or altering an output sound of the skincare device.

13. The skincare and facial scanning system of claim 1, wherein the device setting is a predefined device setting causing the skincare device to operate in a predefined device mode.

14. A skincare and facial scanning method for providing skincare device connectivity and setting configurations, the skincare and facial scanning method comprising:
scanning, by a skincare device comprising a sensor, skin of a user;
depositing, by the skincare device, a cosmetic ink composition onto the skin of the user; and
generating, by a skincare application (app) communicatively coupled to the skincare device, a user-specific electronic analysis based on use of the skincare device on skin of a face portion of the user,
wherein at least a portion of the user-specific electronic analysis is configured for display on a graphic user interface.

15. The skincare and facial scanning method of claim 14,
wherein the skincare device further comprises an identification certificate stored on a memory of the skincare device, and
wherein the identification certificate is configured to uniquely identify the skincare device to the skincare app upon a connection between the skincare device and the skincare app.

16. The skincare and facial scanning method of claim 15, wherein the connection is a persistent connection maintaining connectivity between the skincare device and the skincare app for a plurality of uses of the skincare device.

17. The skincare and facial scanning method of claim 15, further comprising:
pairing the identification certificate of the skincare device to a digital account of the user,
wherein usage or device settings data defining the use or operation of the skincare device is associated with the digital account of the user.

18. The skincare and facial scanning method of claim 14,
wherein usage data defining use of the skincare device is captured over time including at a first time and a second time, and
wherein the user-specific electronic analysis is based on tracking usage data captured at the first time and the second time.

19. The skincare and facial scanning method of claim 17, wherein the usage or device settings data comprises at least one of: (1) an amount of the cosmetic ink composition deposited on the skin of the user; (2) a number of scans of the skin of the user; or (3) a time or a duration of usage of the skincare device by the user.

20. A tangible, non-transitory computer-readable medium storing instructions for providing skincare device connectivity and setting configurations, that when executed by one or more processors cause the one or more processors to:
scan, by a skincare device comprising a sensor, skin of a user;
deposit, by the skincare device, a cosmetic ink composition onto the skin of the user; and
generate a user-specific electronic analysis based on use of the skincare device on skin of a face portion of the user,
wherein at least a portion of the user-specific electronic analysis is configured for display on a graphic user interface.
